(19) United States
(12) Patent Application Publication
(10) Pub. No.: US 2007/0149996 A1
(43) Pub. Date: Jun. 28, 2007

(54) Low profile filter

(75) Inventor: Katherine Coughlin, Ipswich, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/320,035

(22) Filed: Dec. 28, 2005

(51) Int. Cl. A61M 29/00

(52) U.S. Cl. 606/200

(57) ABSTRACT

A low-profile, embolic protection device for collecting particulate debris in a body lumen, wherein an embolic filter is utilized that is expandable and collapsible by push-pull action. A filter first end is axially secured to an elongate inner member and a filter second end is attached to a distal tip of an outer tubular member. The filter is delivered to a treatment site inverted within a distal portion of the outer tubular member. The elongate inner member is slidable within the outer shaft, such that relative longitudinal movement between the elongate inner member and outer shaft reconfigures the filter between its inverted and everted configurations, as well as its expanded and collapsed configurations.

### FIELD OF THE INVENTION

The invention relates generally to intraluminal distal protection devices for capturing particulate in the vessels of a patient. More particularly, the invention relates to a low profile filter for capturing emboli in a blood vessel during an interventional vascular procedure.

### BACKGROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent in the body on a permanent or semi-permanent basis to support weakened or diseased vascular walls to avoid closure or rupture thereof.

These non-surgical interventional procedures often avoid the necessity of major surgical operations. However, one common problem associated with these procedures is the potential release into the bloodstream of atherosclerotic or thrombotic debris that can embolize distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, particles of clot or plaque material can sometimes dislodge from the stenosis during a balloon angioplasty procedure and become released into the bloodstream.

Medical devices have been developed to attempt to deal with the problem created when debris or fragments enter the circulatory system during vessel treatment. One technique includes the placement of a filter or trap downstream from the treatment site to capture embolic debris before it reaches the smaller blood vessels downstream. A filter placed in the patient's vasculature before or during treatment of the vascular lesion can collect embolic debris in the bloodstream.

It is known to attach an expandable filter to a distal end of a guidewire or guidewire-like member that allows the filtering device to be placed in the patient's vasculature. The guidewire allows the physician to steer the filter to a location downstream from the area of treatment. Once the guidewire is in proper position in the vasculature, the embolic filter can be deployed to capture embolic debris. Some embolic filtering devices utilize a restraining sheath to maintain a self-expanding filter in a collapsed configuration. Once the restraining sheath is retracted by the physician withdrawing the proximal end of the sheath extending outside the patient's body, the expandable filter will attempt to transform itself into its fully expanded configuration. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guide wire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. With the physician manipulating a proximal portion of the device outside the patient's body, pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be additionally used as a retrieval catheter at the end of the interventional procedure to reduce the profile of the “push-pull” filter, as due to the embolic particles collected, the filter may still be in a somewhat expanded state. The retrieval catheter may be used to further collapse the filter and/or smooth the profile thereof, so that the filter guidewire may pass through the treatment area without disturbing any stents or otherwise interfering with the treated vessel.

However, regardless of how a distal protection filter is expanded during a procedure, i.e., sheath delivered or by use of a push-pull mechanism, a crossing profile of the collapsed filter is to be at a minimum to reduce interference between the filter and other interventional devices or in-placed stents. As well, a compact filter profile is beneficial in crossing severely narrowed areas of vascular stenosis. Thus, what is needed is a filter that achieves a reduced profile without sacrificing the strength and stability needed for effective embolic capture and retention.

### BRIEF SUMMARY OF THE INVENTION

The present invention is a filtering system for collecting embolic debris in a body lumen. The filtering system includes an outer tubular member having a distal portion and a distal tip, an elongate inner member longitudinally slidable within the outer tubular member, and an embolic filter. The filter has a first end axially secured about the elongate inner member in a first joint, a second end fixedly attached to the distal tip of the tubular member in a second joint, and at least one opening for receiving debris. In the filter's delivery configuration, the first joint is disposed within the outer tubular member's distal portion proximal of the second joint, such that the filter is in an inverted configuration within the outer tubular member's distal portion. Upon positioning of the filter within the body lumen distal of the treatment site, distal movement of the elongate inner member relative to the outer tubular member everts the filter. Continued distal movement of the elongate inner member relative to the outer tubular member draws the first joint through the second joint to fully transform the filter into its extended, everted configuration. Once the filter is everted, proximal movement of the elongate inner member relative to the outer tubular member transforms the filter from its everted configuration into its expanded configuration, such that the filter contacts a wall of the body lumen when fully deployed.

In various embodiments of the present invention, both the elongate inner member and the outer tubular member may be comprised of a hypotube and/or polyimide tubing. Alternatively, the elongate inner member may be comprised of a core wire. The filter may be a braided filter comprised of metallic and/or polymeric filaments.

Another embodiment of the present invention is a method of using a filtering device for distal embolic protection during an interventional procedure within a patient's vessel. The method includes delivering the filtering device, which has an elongate inner member, a hollow outer tubular member and a filter, to a treatment site within a body lumen. During delivery, the filter is held within a distal portion of the outer tubular member in an inverted configuration. The filter is deployed into its expanded configuration by a two-step maneuver. Firstly, the filter is transformed into an everted configuration by distally translating the elongate inner member relative to the outer tubular member to expose and evert the filter distal of the outer tubular member. Secondly, the filter is transformed into an expanded configuration by proximally translating the elongate inner member relative to the outer tubular member, such that an outer surface of the filter is in apposition with the lumen wall for filtering embolic debris from fluid flowing through the vessel during the interventional procedure. After the interventional procedure, a profile of the filter is minimized for removal from the vessel by distally translating the elongate inner member relative to the tubular member to transform the filter into a collapsed configuration.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of a filter system in accordance with an embodiment of the present invention.

FIG. 2 is a partial cross-section of a distal end of the filter system of FIG. 1 within a patient's vascular anatomy with the filter in an inverted configuration within the outer shaft.

FIG. 3 illustrates a distal end of the filter system of FIG. 1 with the filter in its exposed, everted configuration.

FIG. 4 illustrates a distal end of the filter system of FIG. 1 with the filter in its deployed, expanded configuration within the patient's vascular anatomy.

FIG. 5 illustrates a distal end of the filter system of FIG. 1 with the filter in its collapsed configuration prior to removal from the patient's vasculature.

FIG. 6 is a cross-sectional view of a distal end of the filter system of FIG. 1 in accordance with an alternate embodiment of the present invention.

FIG. 7 is a partial cross-section of a distal end of filter system within a patient's vascular anatomy with the filter in an inverted configuration according to another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

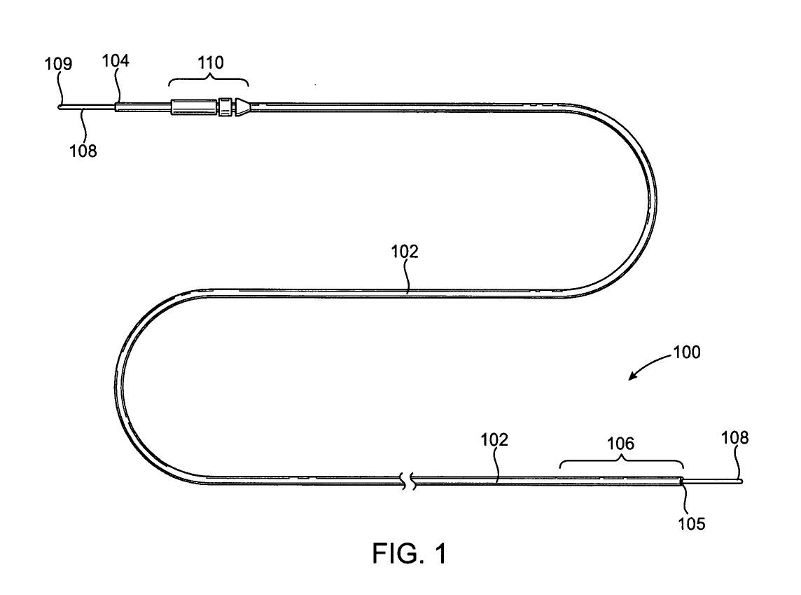

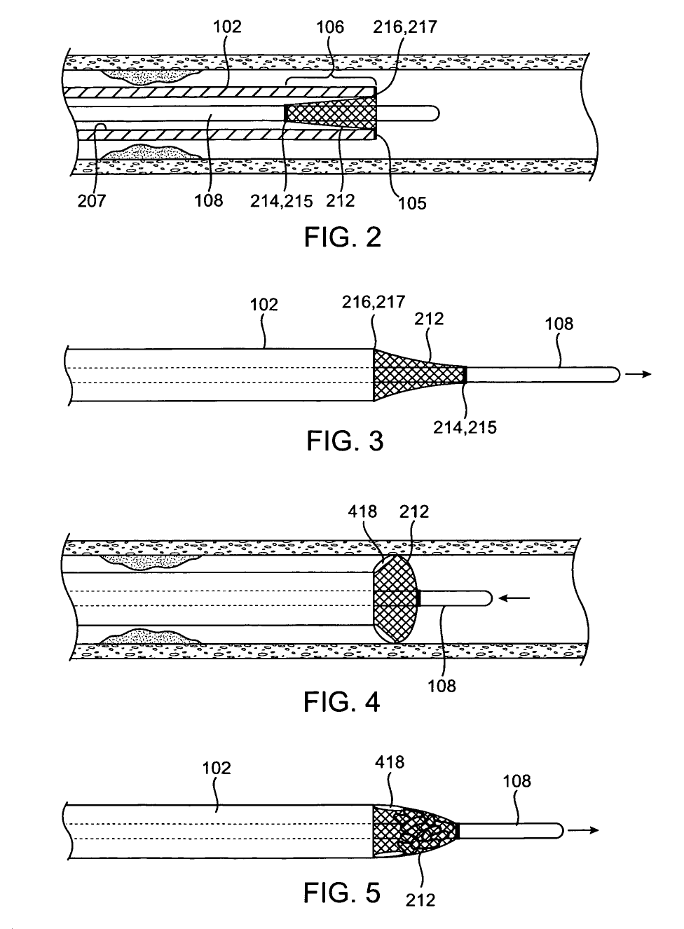

The present invention is a temporary distal protection device for use in minimally invasive procedures, such as vascular interventions or other procedures, where the practitioner desires to capture embolic material that may be dislodged during the procedure. As shown in FIGS. 1 and 2, distal protection device 100, viz, filter system 100, includes an elongate tubular member, or catheter shaft, 102, a core wire 108 slidably extending there through, and a hub 110. Core wire 108 extends within a lumen 207 of tubular member 102 from a proximal end 104 to a distal tip 105 thereof. Tubular member 102 includes a distal portion 106 that accommodates an embolic filter 212 in its delivered configuration. Filter 212 is inverted, or turned inside out, within lumen 207 of distal portion 106, such that filter system 100 has a very low crossing profile during delivery of the device to the treatment site. As such, filter system 100 may be deployed in, for instance, chronic total occlusions (CTOs), or nearly totally occluded vessels.

Embolic filter 212 has a first end 214 and a second end 216. Filter first end 214 is axially secured to core wire 108 in a core wire joint 215, and filter second end 216 is attached to distal tip 105 of tubular member 102 in a distal tip joint 217. Filter joints 215, 217 may be spot welds, laser welds, soldered, brazed, comprised of a bonding sleeve, and/or an adhesive in order to fixedly attach filter ends 214, 216 to core wire 108 and tubular member 102, respectively, as would be apparent to one of ordinary skilled in the relevant art.

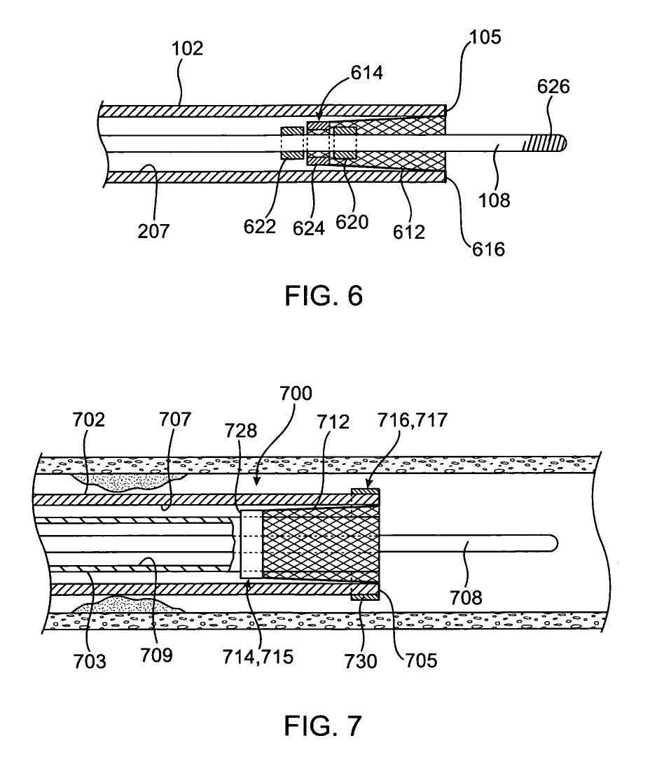

FIG. 6 illustrates an alternate arrangement for joining the filter's first end 614 to core wire 108. As in the previous embodiment, core wire 108 runs coaxially within tubular member 102. A filter 612 in its delivered configuration is positioned within lumen 207, between core wire 108 and tubular member 102. Filter first end 614 is affixed to a cylindrical collar or bearing 624, such that core wire 108 may rotate relative to filter 612 and tubular member 102. Bearing 624 is held in its axial position relative to core wire 108 by stops 620, 622, which are fixedly attached to core wire 108. Filter second end 616 is attached to distal tip 105 of tubular member 102 by any of the methods described for the previous embodiment.

Core wire 108 may be made from a metal, such as nitinol, or a stainless steel wire. In an embodiment of the present invention (not shown), core wire 108 may be tapered at its distal end and/or be comprised of one or more core wire sections. Core wire 108 may be ground down and have several diameters in its profile in order to provide one or more stiffness transitions. Core wire 108 has a proximal end 109 that extends outside of the patient from proximal end 104 of tubular member 102. Core wire 108 may also include a coiled tip portion, such as, coiled tip portion 626 shown in FIG. 6, or may include a flexible coil spring that is formed from a round or flat coil of stainless steel and/or one of various radiopaque alloys, such as platinum, as is well known to those of skill in the art of medical guidewires.

In another embodiment of the present invention, tubular member or catheter shaft 102 may be constructed of multiple shaft components (not shown) of varying flexibility to provide a gradual transition in flexibility. Such a shaft arrangement is disclosed in U.S. Pat. No. 6,706,055, which is incorporated by reference herein in its entirety. In addition, a liner or axial bearings (not shown) as disclosed in the '055 patent may be utilized between core wire 108 and outer shaft 102 in order to facilitate sliding movement there between during expansion and collapse of filter 212. In another embodiment, tubular member 102 may be a hollow tube enabling filter system 100 to also function as a medical guidewire.

Tubular member 102 may include a thin-walled, tubular structure of a metallic material, such as stainless steel, nitinol, or a cobalt-chromium super alloy. Such metallic tubing is commonly referred to as hypodermic tubing or a hypotube. Metallic tubing formed from other alloys, as disclosed in U.S. Pat. No. 6,168,571, which is incorporated by reference herein in its entirety, may also be used in the tubing of the present invention. In the alternative, outer shaft 102 may include tubing made from a thermoplastic material, such as polyethylene block amide copolymer, polyvinyl chloride, polyethylene, polyethylene terephthalate, polyamide, or a thermoset polymer, such as polyimide.

Filter 212 is a braided filter comprised of a plurality of wires or non-metallic filaments that are woven together to form the tubular braided filter. In an embodiment of the present invention, braiding wires or filaments are preferably made from stainless steel, a shape memory material, such as nitinol, a nickel-based super alloy, and/or a suitable polymer. In another embodiment, filter 212 may be formed from a suitable mesh or porous material that collects embolic debris while permitting fluid to flow there through, such as blood flow sufficient for perfusion of body tissues. Such mesh filters and braided filters are disclosed in U.S. Pat. No. 6,346,116 that is incorporated by reference herein in its entirety.

Filter 212 is sized and shaped such that when it is fully deployed, as shown in FIG. 4, its greatest expanded diameter will contact the inner surface of the blood vessel wall into which it is placed. The inner surface contact is preferably maintained over a substantial portion of the expanded circumference to prevent any emboli from escaping past filter 212.

Filter 212 may be self-expanding, meaning that filter 212 has a mechanical memory to return to the expanded, or deployed configuration, once filter 212 is inverted as described below with reference to FIGS. 3 and 4. Such mechanical memory can be imparted to the metal comprising filter 212 by thermal treatment to achieve a spring temper in stainless steel, for example, or to set a shape memory in a susceptible metal alloy, such as nitinol. In such an embodiment of the present invention, it is preferable that at least the majority of braiding wires forming filter 212 be capable of being heat treated into the desired filter shape/component, and such wires should also have sufficient elastic properties to provide the desired self-expanding or self-collapsing features.

Filter system 100 is transformable into its deployed, i.e., expanded, and collapsed configurations by relative movement between first and second ends 214, 216 of filter 212. Filter system 100 is tracked through a patient's vasculature with filter 212 in its inverted configuration within shaft 102, as shown in FIG. 2. Once filter 212 is situated distal of the treatment site, filter 212 is deployed into its expanded configuration in two motions, i.e., in a two-step procedure. Initially, core wire 108 is distally advanced relative to shaft 102. The distal movement of core wire 108 draws core wire joint 215 and filter first end 214 through distal tip joint 217 and filter second end 216 to evert and expose filter 212 distal of shaft 102, as shown in FIG. 3. Accordingly, filter 212 is fully everted, i.e., has its interior side as delivered turned outward to become its exterior side as deployed, through the distal movement of core wire 108 relative to tubular member 102. Core wire 108 is then pulled proximally relative to shaft 102, as represented by the arrow in FIG. 4, to expand filter 212 into contact with the walls of the vessel lumen. In its expanded configuration, proximal filter inlet openings 418 of filter 212 are properly positioned to receive embolic debris. Filter inlet openings 418 are of a shape and size for receiving particulate debris there through, whereas the remaining braided filter openings are sized to collect embolic debris within filter 212 while permitting fluid to flow there through, such as blood flow sufficient for perfusion of body tissues.

Filter 212 is collapsed for removal from the body lumen by once again distally advancing core wire 108 relative to shaft 102, as shown in FIG. 5. Filter system 100 may then be removed from the body lumen in the collapsed and still everted configuration shown in FIG. 5. The collapsed configuration shown in FIG. 5 may be comparable to the everted configuration shown in FIG. 3 or, if a sizable load of embolic debris has been captured, then filter 212 may not collapse that far. In the latter case, as would be apparent to one of ordinary skill in the art, a sheath catheter may be used to assist in the removal of filtering system 100. A suitable sheath catheter for use with the present invention is disclosed in U.S. Pat. No. 6,059,814, which is incorporated by reference herein in its entirety.

Optionally, radiopaque markers (not shown) may be placed on first and second ends 214, 216 of filter 212 to fluoroscopic observation during manipulation thereof. Alternatively, fluoroscopic visualization of the filter may be enhanced when at least one of the filaments includes a wire having enhanced radiopacity compared to conventional non-radiopaque wires suitable for braiding filter 212. Braiding wire having enhanced radiopacity may be made of, or coated with a radiopaque metal such as gold, platinum, tungsten, alloys thereof, or other biocompatible metals having a relatively high X-ray attenuation coefficient compared with stainless steel or nitinol. One or more filaments having enhanced radiopacity may be inter- woven with non-radiopaque wires, or all wires comprising filter 212 may have the same enhanced radiopacity. Alternatively, one or more of braiding wires/braid filaments may comprise a composite wire having a radiopaque core and non-radiopaque layer or casing. Such coaxial, composite wires are referred to as DFT (drawn-filled-tube) wires in the metallic arts, and filters comprising such wires are disclosed in U.S. Pat. No. 6,866,677 B2, which is incorporated by reference herein in its entirety.

A further embodiment of the present invention is shown in FIG. 7 situated within a body lumen, with an embolic filter 712 in its inverted, delivery configuration. Filtering device 700 includes filter 712 attached at a first end 714 to an inner tubular member or shaft 703 and attached at a second end 716 to a distal tip 705 of an outer tubular member or shaft 702. Inner tubular member 703 includes a lumen 709 to slidably accommodate a guidewire 708 therein, where as outer tubular member 702 includes a lumen 707 to slidably accommodate inner tubular member 703 therein. Filter first end 714 is axially secured to inner tubular member 703 in a first joint 715, and filter distal end 716 is attached to distal tip 705 of outer tubular member 702 in a second joint 717. Filter joints 715, 717 may be spot welds, laser welds, soldered, brazed, comprised of a bonding sleeve, and/or an adhesive in order to fixedly attach filter ends 714, 716 to inner and outer and tubular members 703, 702, respectively, as would be apparent to one of ordinary skilled in the relevant art. Alternatively, filter first end 714 may be rotatably secured to inner tubular member 703 as described above regarding filter first end 614. In this embodiment, radiopaque marker bands 728, 730 are secured around first and second joints 715, 717 respectively, to delimitate filter ends 714, 716 during fluoroscopic observation of filter 712.

Filter system 700 is transformable between its deployed, i.e., expanded, and collapsed configurations by relative movement between first and second ends 714, 716 of filter 712. Filter system 700 is tracked through a patient's vasculature over guidewire 708 with filter 712 in its inverted configuration within lumen 707 of outer shaft 702, as shown in FIG. 7. Once filter 712 is situated distal of the treatment site, filter 712 is deployed into its expanded configuration in a two-step process, similar to the process described above for the embodiment shown in FIGS. 3 and 4. Initially, inner shaft 703 is distally advanced relative to outer shaft 702. The distal movement of inner shaft 703 draws first joint 715 and filter first end 714 through second joint 717 and filter second end 716 to evert and expose filter 712 distal of outer shaft 702. Accordingly, filter 712 is fully everted, i.e., has its interior side as delivered turned outward to become its exterior side as deployed, through the distal movement of inner shaft 703 relative to outer shaft 702. Inner shaft 703 is then pulled proximally relative to outer shaft 702 to expand filter 712 into apposition with the wall of the lumen.

Similarly to the embodiment shown in FIG. 5, filter 712 is collapsed for removal from the body lumen by once again distally advancing inner shaft 703 relative to outer shaft 702, so that filter 712 is removed from the body lumen in its collapsed and everted configuration. Inner tubular member 703 and outer tubular member 702 may be of any construction or material previously described with reference to tubular member 102. Filter 712 may also be of any construction or material previously described with reference to filter 212.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A filtering device for collecting debris in a body lumen, comprising:
a hollow tubular member having a distal portion and a distal tip;
a core wire slidable within the tubular member; and
a braided filter having a first end axially secured about the core wire in a core wire joint, a second end fixedly attached to the distal tip of the tubular member in a distal tip joint, and an opening for receiving debris, wherein when the filtering device is in its delivery configuration the core wire joint is disposed within the tubular member distal portion proximal of the distal tip joint, such that the filter is in an inverted configuration within the tubular member distal portion.

2. The filtering device of claim 1, wherein the filter is transformable into an everted configuration by distal movement of the core wire relative to the tubular member.

3. The filtering device of claim 2, wherein the core wire joint is moveable through the distal tip joint by the distal movement of the core wire relative to the tubular member.

4. The filtering device of claim 3, wherein the filter is transformable from the everted configuration into an expanded configuration, wherein the filter contacts the body lumen, by proximal movement of the core wire relative to the tubular member.

5. The filtering device of claim 1, wherein the tubular member is comprised of a hypotube.

6. The filtering device of claim 5, wherein the filter is transformable into an everted configuration by distal movement of the core wire relative to the hypotube.

7. The filtering device of claim 1, wherein the tubular member is comprised of polyimide tubing.

8. The filtering device of claim 1, wherein the core wire joint permits the first end of the filter to rotate relative to the core wire.

9. The filtering device of claim 1, wherein the second end of the filter is fixedly attached to the distal tip of the tubular member in the distal tip joint by an attachment means selected from a group consisting essentially of an adhesive, soldering and brazing.

10. A method of using a filtering device for distal protection during an interventional procedure comprising:
delivering the filtering device having an elongate inner member, a hollow outer tubular member and a filter to a treatment site within a body lumen, wherein during delivery the filter is held within a distal portion of the tubular member in an inverted configuration;
distally translating the elongate inner member relative to the outer tubular member to expose and transform the filter into an everted configuration distal of the outer tubular member; and
proximally translating the elongate inner member relative to the tubular member to deploy the filter into an expanded configuration, such that an outer surface of the filter is in apposition with the wall of the body lumen for filtering embolic debris during the interventional procedure.

11. The method of claim 10 further comprising:
minimizing a profile of the filter for removal from the body lumen by distally translating the elongate inner member relative to the outer tubular member to transform the filter into a collapsed configuration.

12. The method of claim 10, wherein a first end of the filter is axially secured to the elongate inner member in a first joint and a second end of the filter is fixedly attached to a distal tip of the outer tubular member in a distal tip joint, such that distal translation of the elongate inner member relative to the outer tubular member draws the first joint through the distal tip joint to transform the filter into the everted configuration.

13. The method of claim 12, wherein the elongate inner member is a solid core wire.

14. The method of claim 12, wherein the elongate inner member is a hollow tubular member.

15. The method of claim 14, wherein at least one of the inner and outer hollow tubular members is a hypotube.

16. A filtering device for collecting debris in a body lumen, comprising:
an outer tubular member having a lumen and a distal tip;
an inner tubular member slidable within the outer tubular member lumen; and
a filter having a first end axially secured about the inner tubular member in a first joint, a second end fixedly attached to the distal tip of the outer tubular member in a distal tip joint, and an opening for receiving debris, wherein when the filtering device is in its delivery configuration the first joint is disposed within the outer tubular member lumen proximal of the distal tip joint, such that the filter is in an inverted configuration within the outer tubular member lumen.

17. The filtering device of claim 16, wherein the filter is transformable into an everted configuration by distal movement of the inner tubular member relative to the outer tubular member.

18. The filtering device of claim 17, wherein the first joint is drawable through the distal tip joint by the distal movement of the inner tubular member relative to the outer tubular member.

19. The filtering device of claim 18, wherein the filter is transformable from the everted configuration into an expanded configuration, such that the filter is in apposition with a wall of the body lumen, by proximal movement of the inner tubular member relative to the outer tubular member.

20. The filtering device of claim 16, wherein at least one of the inner and outer tubular members is comprised of a hypotube.

21. The filtering device of claim 16, wherein the inner tubular member includes a lumen for accommodating a guidewire there through.

22. The filtering device of claim 21, wherein at least one of the inner and outer tubular members is comprised of polyimide tubing.

23. The filtering device of claim 16, wherein the first joint permits the first end of the filter to rotate relative to the inner tubular member.
